(19) United States
(12) Patent Application Publication
(10) Pub. No.: US 2025/0276014 A1
(43) Pub. Date: Sep. 4, 2025

(54) GENETICALLY ENGINEERED INNATE LYMPHOID CELLS FOR ENHANCING LIFESPAN AND/OR TREATING CANCERS

(71) Applicant: ACADEMIA SINICA, Taipei (TW)

(72) Inventors: Che-Kun James SHEN, Taipei City (TW); Chun-Hao HUNG, Tainan City (TW); Shih-Ling HUANG, Yunlin County (TW); Keh-Yang WANG, New Taipei City (TW); Jing-Ping WANG, Taipei City (TW); Zheng-Sheng LAI, Chuanghua County (TW)

(21) Appl. No.: 18/858,109

(22) PCT Filed: Apr. 20, 2023
(86) PCT No.: PCT/US23/66011

§ 371 Date: Oct. 18, 2024

Related U.S. Application Data
(60) Provisional application No. 63/363,332, filed on Apr. 21, 2022.

(51) Int. Cl. A61K 35/17; A61P 35/00; C07K 14/47; C12N 5/0783

(52) U.S. Cl. CPC A61K 35/17; A61P 35/00; C07K 14/47; C12N 5/0646; C12N 2501/2302; C12N 2501/2315; C12N 2510/00; C12N 2517/02

(57) ABSTRACT

The disclosure is related to the field of innate lymphoid cells (ILCs). Particularly, the disclosure pertains to genetically engineered NK cells carrying a modified Eklf gene encoding a modified EKLF polypeptide.

### CROSS-REFERENCE TO RELATED APPLICATIONS

This application is a 371 National Stage application based upon International Patent Application No. PCT/US23/66011, filed Apr. 20, 2023, which claims the benefit of priority of U.S. Provisional Application No. 63/363,332, filed 21 Apr. 2022, the contents of which are incorporated by reference herein in their entirety for any purpose.

### SEQUENCE LISTING

The instant application contains a Sequence Listing which is submitted electronically in .xml format and is hereby incorporated by reference in its entirety. The .xml copy, created on Apr. 20, 2023, is named “G4590-16100NP_20241015_SeqListing.xml” and is 4,096 bytes in size.

### TECHNICAL FIELD

The present disclosure is related to the field of innate lymphoid cells (ILCs). Particularly, the disclosure pertains to genetically engineered NK cells carrying a modified Eklf gene encoding a modified EKLF polypeptide.

### BACKGROUND

Innate lymphoid cells (ILCs) are innate immune cells, which are derived from common lymphoid progenitors (CLPs). ILCs can be divided into five groups: natural killer (NK) cells, ILC1s, ILC2s, ILC3s, and lymphoid tissue inducer (LTi) cells. ILC1 and NK cell lineages are involved in Type 1 immunity such as macrophage activation, cytotoxicity, oxygen radicals. ILC2 are involved in Type 2 immunity such as mucus production, alternative macrophage activation, and extracellular matrix/tissue repair. ILC3 are involved in Type 3 immunity such as phagocytosis and antimicrobial peptides. LTi are involved in the formation of secondary lymphoid structures.

The study of longevity genes is a novel developing field. However, very few of these genes have been identified and even less is understood about how they contribute to longevity. A few of the polymorphisms associated with long life spans are found in the APOE, FOXO3, and CETP genes, but they are not found in all individuals with exceptional longevity. Wu et al. (Human molecular genetics 21, 3956-3968) described that overexpression of Cisd2 in mice extends their lifespan and ameliorates age-associated degeneration of the skin, skeletal muscles and neurons. WO 2016036727 provides a non-human transgenic animal comprising one or more modified Erythroid Kruppel-like factor (EKLF) genes encoding a modified EKLF polypeptide comprising one or more amino acid modifications as compared to a wild-type EKLF polypeptide. The genetically altered EKLF mice display extended lifespan, extended healthspan, and resistance to cancer incidence and/or metastasis.

Both innate (natural) and adaptive (acquired) immune responses decline with advancing age. In terms of reduced tolerance to stress in the elderly, development of a powerful responders such as NK-cells may be especially important. The role of innate lymphoid cells and longevity genes on the escape of age-related diseases with subsequent healthy aging and longevity remains unknown.

### SUMMARY

The present disclosure provides a method of increasing longevity and/or treating cancer in a subject, comprising administering an effective amount of genetically engineered innate lymphoid cells to the subject, wherein the genetically engineered innate lymphoid cells express a modified EKLF polypeptide comprising an amino acid substitution at a sumoylation site of a wild-type EKLF polypeptide having the amino acid sequence of SEQ ID NO: 1 or SEQ ID NO: 2.

In some embodiments, the genetically engineered innate lymphoid cells are natural killer (NK) cells.

In some embodiments, the genetically engineered innate lymphoid cells are prepared by the steps of:
(a) providing a transgenic animal expressing the modified EKLF polypeptide;
(b) isolating innate lymphoid cells from the transgenic animal; and
(c) expanding the innate lymphoid cells with a cytokine selected from the group consisting of IL-2, IL-12, IL-15, IL-18 and IL-21; and optionally
(d) contacting the innate lymphoid cells with an irradiated feeder cells.

In some embodiments, the sumoylation site corresponds to lysine at position 54 of a wild type human EKLF, or corresponds to lysine at position 74 of a wild type mouse EKLF.

In some embodiments, the method as described above further comprises administering long-term hematopoietic stem cells (LT-HSC) expressing a modified EKLF polypeptide to the subject, wherein the modified EKLF polypeptide comprises a substitution of K54R in the wild type human EKLF, or a substitution of K74R in the wild type mouse EKLF.

In some embodiments, the NK cells are (i) CD56^{bright} NK cells expressing CD62L, CCR7 and CXCR4; (ii) CD56^{dim} NK cells expressing CXCR1, CX3CR1 and ChemR23; (iii) express NK1.1 (CD161) and NKp44 or Nkp46; (iv) express CD49b; or (v) express CD45 and CD56. In some embodiments, the innate lymphoid cells (i) do not express the innate lymphoid

cells do not express CD117, Sca-1, and CD90.1 (Thy1.1) (ii) express CRTH2, KLRG1, SST2, CD25, CD44 and CD161; (iii) express RORγt, NKp30 and CD56; or (iv) express c-Kit, CCR6, CD25, CD90, CD127, and OX40L.

In some embodiments, the modified human EKLF polypeptide comprises a substitution of the lysine (K) residue corresponding to position 54 of the wild type human EKLF with an arginine (R) or with another amino acid that confers tumor resistance and healthy longevity.

In some embodiments, the modified EKLF polypeptide is a modified mouse EKLF polypeptide comprises an amino acid substitution at position 68 of the full length wild-type mouse EKLF polypeptide.

In some embodiments, the genetically engineering innate lymphoid cells comprise transducing the innate lymphoid cells with a viral vector encoding the modified EKLF polypeptide.

In some embodiments, the genetically engineering innate lymphoid cells comprise using clustered regularly interspaced short palindromic repeats (CR1SPR) or CR1SPR associated proteins (Cas) technique.

In some embodiments, the method as described above comprises administering 5×10⁶ to 5×10⁸ genetically engineered innate lymphoid cells per kilogram to the subject.

In some embodiments, the cancer is liver cancer, colon cancer, breast cancer, prostate cancer, hepatocellular carcinoma, skin cancer, lung cancer, glioblastoma, brain cancer, hematopoietic malignancies, retinoblastoma, renal cell carcinoma, head and neck cancer, cervical cancer, pancreatic cancer, esophageal cancer, or squama cell carcinoma.

The present disclosure further provides an in vitro method of producing genetically engineered innate lymphoid cells, comprising:
(a) providing a transgenic animal expressing a modified human EKLF polypeptide comprising an amino acid substitution at the sumoylation site that corresponds to lysine at position 54 of the wild type human EKLF having the amino acid sequence of SEQ ID NO: 1, or a modified mouse EKLF polypeptide comprising an amino acid substitution at the sumoylation site that corresponds to lysine at position 74 of the wild type mouse EKLF having the amino acid sequence of SEQ ID NO:2; and
(b) isolating innate lymphoid cells from the genetically engineered animal.

In some embodiments, the method as described above further comprises expanding the innate lymphoid cells with a cytokine selected from the group consisting of IL-2, IL-12, IL-15, IL-18 and IL-21.

In some embodiments, the method as described above further comprises depleting CD3⁺ cells using magnetic beads coated with an anti-CD3 antibody.

In some embodiments, the method as described above further comprises contacting the innate lymphoid cells with an irradiated feeder cells.

In some embodiments, the irradiated feeder cells are EBV-transformed lymphoblastoid cell lines, or leukemia cell lines modified to express a membrane-bound form of IL-15 and 41BB ligand.

The present disclosure further provides an engineered innate lymphoid cell comprises a gene encoding a modified EKLF polypeptide, wherein the modified EKLF polypeptide comprises an amino acid substitution at a sumoylation site of a wild-type EKLF polypeptide, wherein the sumoylation site corresponds to lysine at position 54 of a wild type human EKLF having the amino acid sequence of SEQ ID NO: 1, or corresponds to lysine at position 74 of a wild type mouse EKLF having the amino acid sequence of SEQ ID NO: 2.

### BRIEF DESCRIPTION OF THE DRAWINGS

FIGS. 1A to 1E show expansion of mouse NK cells and in vitro cancer cell killing capability of mouse NK (K74R) cells. Mouse NK cells were sorted from spleen and expanded by IL-15 (50 ng/ml) for 6 days. IL-2 (200 U/ml) were added to prime the NK cells at day 5. After 6 days expansion, NK cells were co-cultured with B16F10 (labeled with CFSE) at different E (effect):T (target) ratio, e.g., 1:1, 3:1 and 9:1. for 4 hours (FIG. 1A). Mouse Eklf (K74R) NK cells show higher cancer cell cytotoxicity than wild-type NK cells. Specific lysis in B16-F10 (Melanoma) (FIG. 1B), 4T1 (Breast cancer) (FIG. 1C), LLC1 (Lung cancer) (FIG. 1D) and YAC-1 (Lymphoma) (FIG. 1E) were analyzed by flow cytometry.

FIGS. 2A to C show isolation and expansion of mouse NK cells and capability of mouse NK (K74R) cells to inhibit tumor growth in vivo by mouse Eklf (K74R) NK cells. Mouse NK cells were sorted from spleen and expanded for 7 days. Expanded mouse NK cells (2×10⁶/mouse) were injected into B16-F10 bearing mice on day 4. The mice were then subjected to in vivo imaging system (IVIS) on day 4, 7, 11, 14 (FIG. 2A). Mouse NK (K74R) cells show higher capability to inhibit tumor growth in vivo. Cultured B16-F10 cancer cells were injected into the NSG mice on day 0 and then monitored for tumor growth on day 4, 7, 11, 14. N>3/group. **p<0.01; #, p<0.0001 (FIGS. 2B and 2C).

FIG. 3 shows administration of bone marrow mononuclear cells (BMMNC) (K74R) prolongs the lifespan of recipient wild-type mice. The bone marrow mononuclear cells of 2 month-old WT or Eklf (K74R) mice were injected into the vein of 2 month-old WT recipient mice. The medium survival curve of mice receiving BMMNC from WT mice is 29 months, while that of mice receiving BMMNC transplantation from the Eklf (K74R) mice is 33 months. N=10, p<0.05.

### DETAILED DESCRIPTION

For convenience, certain terms employed in the context of the present disclosure are collected here. Unless defined otherwise, all technical and scientific terms used herein have the same meaning as commonly understood by one of the ordinary skill in the art to which this disclosure belongs.

The singular forms “a”, “and”, and “the” are used herein to include plural referents unless the context clearly dictates otherwise.

### Definitions

As used herein, the term “express”, “expression” or “expressing” is intended to refer to transcription of a gene when a condition is met, resulting in the generation of mRNA and usually encoded protein. Expression can be achieved or performed naturally by the cell (i.e., without artificially intervention) or may be achieved or performed artificially (i.e., with the involvement of artificially intervention, such as by the use of promoters regulated by the use of a chemical agent). The expression may also be initiated by a recombination event triggered by a site-specific recombinase, such as by Cre-mediated recombination. Expression may be measured by measuring mRNA transcribed from the gene or by measuring protein encoded by the gene.

As used herein, the term “nucleic acid” refers to polynucleotides such as deoxyribonucleic acid (DNA) and where appropriate, ribonucleic acid (RNA). Nucleic acids include but are not limited to single-stranded and double-stranded polynucleotides. Illustrative.

As used herein, the term “vector” refers to a nucleic acid molecule capable of transporting another nucleic acid to which it has been linked. The term “expression vector” refers to a vector comprising a promoter operably linker to a nucleic acid in a manner allowing expression of the operably linked nucleic acid. Vectors or expression vectors as used herein thus include plasmids or phages capable of synthesizing the subject protein encoded by the respective recombinant gene carried by the vector. Vectors or expression vectors also include viral-based vectors capable of introducing a nucleic acid into a cell, e.g., a mammalian cell. Certain vectors are capable of autonomous replication and/or expression of nucleic acids to which they are linked.

The term “wild-type” refers to a gene or gene product that has the characteristics of that gene or gene product when isolated from a naturally-occurring source. A wild-type gene or gene product (e.g., a polypeptide) is that which is most frequently observed in a population and is thus arbitrarily designed the “normal” or “wild-type” form of the gene.

As used herein, the term “transfection” refers to the introduction of nucleic acid, e.g., an expression vector, into a recipient cell by nucleic acid mediated gene transfer. “Transformation” refers to a process in which a cell's genotype is changed as the result of the cellular uptake of exogenous DNA or RNA, and the transformed cell expresses a desired heterologous protein.

As used herein, the tem “CR1SPR,” “CR1SPR system” or “CR1SPR nuclease system” and their grammatical equivalents can include a non-coding RNA molecule (e.g., guide RNA) that binds to DNA and Cas proteins (e.g., Cas9) with nuclease functionality (e.g., two nuclease domains).

As used herein the term “transgene” refers to a nucleic acid sequence which is partly or entirely heterologous, i.e., foreign, to the transgenic animal or cell into which it is introduced, or is homologous to an endogenous gene of the transgenic animal or cell into which it is introduced, but which is designed to be inserted, or is inserted, into the animal's genome in such a way that the genome of the cell to which it is inserted is altered. A transgene can be operably linked to one or more transcriptional regulatory sequences and any other nucleic acid, such as introns, that may be necessary for optimal expression of a selected nucleic acid. Therefore, the term “transgenic” is used herein as an adjective to describe the property of an animal or a construct, of harboring a transgene. For example, “a transgenic animal” is a non-human animal, preferably a non-human mammal, more preferably, a rodent, in which one or more of the cells of the animal contain heterologous nucleic acid introduced by way of human intervention, such as by transgenic techniques well known in the art, including gene knock-in techniques. The nucleic acid is introduced into the cell, directly or indirectly by introduction into a precursor of the cell, via deliberate genetic manipulation, such as by microinjection or by infection with a recombinant virus. Transgenic animals include, but are not limited to, knock-in animals.

As used herein, the term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” “polymorphism,” and “variant” refer to a gene or gene product that displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “polypeptide,” “peptide” and “protein” are used interchangeably herein to refer to a polymer of amino acid residues.

As used herein, the term “mammal” refers to all members of the class Mammalia, including humans, primates, domestic and farm animals, such as rabbit, pig, sheep, and cattle; as well as zoo, sports or pet animals; and rodents, such as mouse and rat. The term “non-human mammal” refers to all members of the class Mammalis except human.

As used herein, the term “subject” refers to an animal including the human species that may benefit from the method of the present disclosure. The term “subject” intended to refer to both the male and female gender unless one gender is specifically indicated. Accordingly, the term “subject” comprises any mammal which may benefit from the treatment method of the present disclosure.

As used herein, the term “transplantation” and variations thereof refers to the insertion of a transplant (also called graft) into a recipient, whether the transplantation is syngeneic (where the donor and recipient are genetically identical), allogeneic (where the donor and recipient are of different genetic origins but of the same species), or xenogeneic (where the donor and recipient are from different species).

As used herein, the term “donor” refers to an animal, preferably a mammal that is the nature source of the bone marrow cells. The donor can be a healthy mammal, that is, a mammal that is not suffering from any obvious disease. Alternatively, the donor can be a mammal suffering from a disease (e.g., cancer). A recipient is an animal, preferably a mammal, receiving the bone marrow cells from a donor. The recipient can be a healthy mammal, that is, a mammal that is not suffering from any obvious disease. Alternatively, the recipient can be a mammal suffering from a disease (e.g., cancer). According to embodiments of the present disclosure, the donor and the recipient can be the same mammal.

As used herein, the term “an effective amount” as used herein refers to an amount effective, at dosages, and for periods of time necessary, to achieve the desired result with respect to the treatment of a disease.

As used herein, the term “treatment” as used herein is intended to mean obtaining a desired pharmacological and/or physiologic effect, e.g., delaying or inhibiting cancer occurrence, growth, or metastasis, or ameliorating injury to an organ. The effect may be prophylactic in terms of completely or partially preventing or inhibiting occurrence of a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., a cancer or heart failure) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

As used herein, the term “administered”, “administering” or “administration” are used interchangeably herein to refer a mode of delivery, including, without limitation, intraveneously, intramuscularly, intraperitoneally, intraarterially, intracranially, or subcutaneously administering an agent (e.g., a compound or a composition) of the present disclosure.

As used herein, the term “an effective amount” as used herein refers to an amount effective, at dosages, and for periods of time necessary, to achieve the desired result with respect to the treatment of a disease or condition, such as aging. For example, in the treatment of a cancer, innate lymphoid cells which decrease, inhibit, prevent, delay or suppress or arrest any symptoms of the cancer would be effective. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two or more doses in a suitable form to be administered at one, two or more times throughout a designated time period.

As used herein, the term “cell surface marker” means that the subject cell has on its cellular plasma membrane a protein, an enzyme or a carbohydrate capable of binding to an antibody and/or digesting an enzyme substrate. The cell surface markers are recognized in the art to serve as identifying characteristics of particular types of cells.

As used herein, the term “enhancing longevity” “increasing longevity” and “life-extension” are used interchangeably herein and refer to a delay of the normal aging process and/or prolonging the lifespan of an animal, e.g., an animal suffering from a life-threatening disorder (e.g., a cancer or tumor). Preferably, the longevity is due to an extension of the mature life phase, as opposed to an extension of the immature life phase, and is resulted from being treated by the present method.

The present disclosure identifies a technical problem with regard to the distinct property between hematopoietic stem cells (HSCs) and their downstream cells. Specifically, in contrast to T cells and HSCs, NK cells are notoriously difficult to transduce. Efficiency of genetic modification of NK cells to possess a transgene has proven challenging and less efficient than other cells of the HSCs. As NK cells are among the first-line responders to exogenous antigens such as viral infection, their endurance against transduction is considered not unexpected. NK cells express high level of pattern recognition receptors (PRRs), which are activated to respond to pathogen-associated molecular patterns (PAMPs) and danger-associated molecular patterns (DAMPs). Without being limited by theory, it is believed that high level expression of PRRs is one of the reasons causing endurance against transduction. The result is poor NK cell viability and low transduction rates, which hinder the efficacy for therapeutic use.

### Innate Lymphoid Cells

In some embodiments, the innate lymphoid cells are isolated from any source, including, but not limited to, bone marrow, placenta, cord blood, placental blood, peripheral blood, spleen, or liver. In some embodiments, the innate lymphoid cells are NK cells, ILC1s, ILC2s, ILC3s, and lymphoid tissue inducer (LTi) cells. The phenotypic markers of the innate lymphoid cells are shown in Table 1.

TABLE 1

| Type | Phenotypic Markers |
|---|---|
| NK | CD62L, CCR7, CXCR4, CXCR1, CX3CR1, ChemR23, NK1.1 |
| cells | (CD161), NKp44, Nkp46, CD45, CD49b and CD56. |
| ILC1s | NKp44, NKp46 and CD127 |
| ILC2s | CRTH2, KLRG1, SST2, CD25, CD44 and CD161 |
| ILC3s | RORγt, NKp30 and CD56 |
| LTi | c- Kit, CCR6, CD25, CD90, CD127 and OX40L. |
| cells | |

### NK Cell Expansion

In some embodiments, the NK cells are expanded in vitro by contacting them with combinations of cytokines; wherein the cytokines are selected from the group consisting of IL-2, IL-12, IL-15, IL-18 and IL-21. In some embodiments, the NK cells are expanded in vitro by contacting them with the combinations of cytokines and feeder cells; wherein the cytokines are selected from the group consisting of IL-2, IL-12, IL-15, IL-18 and IL-21; and wherein the feeder cells are EBV-transformed lymphoblastoid cell lines, or leukemia cell lines modified to express a membrane-bound form of IL-15 and 41BB ligand. In some embodiments, the NK cells are expanded in vitro by contacting them with the combinations of cytokines, antibodies and feeder cells; wherein the cytokines are selected from the group consisting of IL-2, IL-12, IL-15, IL-18 and IL-21; wherein the feeder cells are EBV-transformed lymphoblastoid cell lines, or leukemia cell lines modified to express a membrane-bound form of IL-15 and 41BB ligand; and wherein the antibodies; and wherein the antibodies are anti-CD56 antibody and anti-CD3 antibody.

During expansion, NK cells were provided with single cytokine or combinations of cytokines every day. Partial medium was replaced every day by fresh medium containing the corresponding single cytokine or combinations of cytokines for 4 days to 10 days.

### EKLF Polypeptide

Homo sapiens Kruppel-like factor 1 (erythroid), mRNA (cDNA clone MGC: 34237 IMAGE: 5201847) was shown in GenBank as Accesion No. BC033580.1 (www.ncbi.nlm.nih.gov/nuccore/BC033580.1).

In some embodiments, the modified EKLF polypeptide comprises an amino acid substitution at a sumoylation site of a wild-type EKLF polypeptide. In some embodiments, the wild-type EKLF polypeptide is a wild-type human EKLF polypeptide having the amino acid sequence of SEQ ID NO: 1 as shown in Table 1. In some embodiments, the wild-type EKLF polypeptide is a wild-type mouse EKLF polypeptide having the amino acid sequence of SEQ ID NO: 2 as shown in Table 2.

TABLE 2

| Source | Amino acid sequence of Wild-type EKLF polypeptide |
|---|---|
| Homo sapiens | MATAETALPS ISTLTALGPF PDTQDDFLKW WRSEEAQDMG |
| | 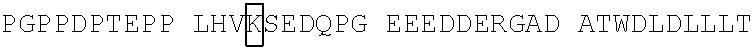 |
| | NFSGPEPGGA PQTCALAPSE ASGAQYPPPP ETLGAYAGGP |
| | GLVAGLLGSE DHSGWVRPAL RARAPDAFVG PALAPAPAPE |
| | PKALALQPVY PGPGAGSSGG YFPRTGLSVP AASGAPYGLL |
| | SGYPAMYPAP QYQGHFQLFR GLQGPAPGPA TSPSFLSCLG |
| | PGTVGTGLGG TAEDPGVIAE TAPSKRGRRS WARKRQAAHT |
| | CAHPGCGKSY TKSSHLKAHL RTHTGEKPYA CTWEGCGWRF |
| | ARSDELTRHY RKHTGQRPFR CQLCPRAFSR SDHLALHMKR |
| | HL (SEQ ID NO: 1) |
| Mus musculus | MRQKRERRPE VQGGHQPAMA SAETVLPSIS TLTTLGQFLD |
| | 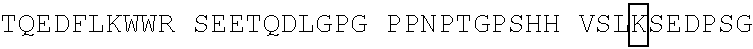 |
| | EDDERDVTCA WDPDLFLTNF PGSESPGTSR TCALAPSVGP |
| | VAQFEPPESL GAYAGGPGLV TGPLGSEEHT SWAHPTPRPP |
| | APEPFVAPAL APGLAPKAQP SYSDSRAGSV GGFFPRAGLA |
| | VPAAPGAPYG LLSGYPALYP APQYQGHFQL FRGLAAPSAG |
| | PTAPPSFLNC LGPGTVATEL GATAIAGDAG LSPGTAPPKR |
| | SRRTLAPKRQ AAHTCGHEGC GKSYTKSSHL KAHLRTHTGE |
| | KPYACSWDGC DWRFARSDEL TRHYRKHTGH RPFCCGLCPR |
| | AFSRSDHLAL HMKRHL (SEQ ID NO: 2) |

### Sumoylation

Small Ubiquitin-like Modifier (“SUMO”) proteins are a family of small proteins that are covalently attached to and detached from other proteins in cells to modify their function. This process is called sumoylation, which is a post-translational modification involved in various cellular processes, such as nuclear-cytosolic transport, transcriptional regulation, apoptosis, protein stability, response to stress, and progression through the cell cycle. In some embodiments, the genetically engineered innate lymphoid cells express a modified EKLF polypeptide comprising an amino acid substitution at a sumoylation site of a wild-type EKLF polypeptide. In some embodiments, the sumoylation site corresponds to lysine at position 54 of a wild-type human EKLF, or corresponds to lysine at position 74 of a wild-type mouse EKLF.

### Method for the Treatment of Cancer

In some embodiments, the method for treating cancer comprises administering an effective amount of genetically engineered innate lymphoid cells to the subject. In some embodiments, the genetically engineered innate lymphoid cells express a modified EKLF polypeptide. In some embodiments, the modified EKLF polypeptide comprises an amino acid substitution at a sumoylation site of a wild-type EKLF polypeptide. In some embodiments, the modified EKLF polypeptide is a modified human EKLF polypeptide having the amino acid sequence of SEQ ID NO: 1. In some embodiments, the modified EKLF polypeptide is a modified mouse EKLF polypeptide having the amino acid sequence of SEQ ID NO: 2.

In some embodiments, the cancer is liver cancer, colon cancer, breast cancer, prostate cancer, hepatocellular carcinoma, skin cancer, hematopoietic malignancy, lung cancer, brain cancer, bone cancer, renal cell carcinoma, head and neck cancer, cervical cancer, pancreatic cancer or esophageal cancer.

In some embodiments, the skin cancer includes, but is not limited to, basal cell carcinoma, squamous cell carcinoma, squamous cell skin cancer, skin adnexal tumors, melanoma, merkel cell carcinoma, or keratoacanthoma.

In some embodiments, the hematopoietic malignancy includes, but is not limited to, acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, Hepatosplenic T-cell lymphoma, Hodgkin's lymphoma, intravascular large B-cell lymphoma, large granular lymphocytic leukemia, Lymphoplasmacytic lymphoma, lymphomatoid granulomatosis, mantle cell lymphoma, marginal zone B-cell lymphoma, mast cell leukemia, mediastinal large B cell lymphoma, multiple myeloma, plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sézary syndrome, splenic marginal zone lymphoma, or T-cell prolymphocytic leukemia.

In some embodiments, the lung cancer includes, but is not limited to, adenocarcinoma of the lung, bronchial adenomas/carcinoids, small cell lung cancer, mesothelioma, non-small cell lung cancer, non-small cell lung carcinoma, pleuropulmonary blastoma, laryngeal cancer, thymoma and thymic carcinoma, or squamous-cell carcinoma of the lung.

In some embodiments, the brain cancer includes, but is not limited to, gliomas, meningiomas, pituitary adenomas and nerve sheath tumors, preferably anaplastic astrocytoma, astrocytoma, central neurocytoma, choroid plexus carcinoma, choroid plexus papilloma, choroid plexus tumor, dysembryoplastic neuroepithelial tumour, ependymal tumor, fibrillary astrocytoma, giant-cell glioblastoma, glioblastoma multiforme (GBM), gliomatosis cerebri, gliosarcoma, hemangiopericytoma, medulloblastoma, medulloepithelioma, meningeal carcinomatosis, neuroblastoma, neurocytoma, oligoastrocytoma, oligodendroglioma, optic nerve sheath meningioma, pediatric ependymoma, pilocytic astrocytoma, pinealoblastoma, pineocytoma, pleomorphic anaplastic neuroblastoma, pleomorphic xanthoastrocytoma, primary central nervous system lymphoma, sphenoid wing meningioma, subependymal giant cell astrocytoma, subependymoma and trilateral retinoblastoma.

In some embodiments, the bone cancer includes, but is not limited to osteoma, osteoid osteoma, osteochondroma, osteoblastoma, enchondroma, giant cell tumor of bone, aneurysmal bone cyst, fibrous dysplasia of bone, osteosarcoma, chondrosarcoma, Ewing's sarcoma and fibrosarcoma.

In some embodiments, the cancer is melanoma, breast cancer, lung cancer or lymphoma.

Dosing regimen of Innate Lymphoid Cells

In one embodiment, the genetically engineered innate lymphoid cells are administered at a dose of about 1.0×10⁵ to about 5.0×10⁶ viable cells/kg, about 0.2×10⁶ to 1.8×10⁶ viable cells/kg, about 0.2×10⁶ to 1.6×10⁶ viable cells/kg, about 0.2×10⁶ to 1.4×10⁶ viable cells/kg, about 0.2×10⁶ to 1.2×10⁶ viable cells/kg, about 0.2×10⁶ to 1.0×10⁶ viable cells/kg, about 0.2×10⁶ to 0.8×10⁶ viable cells/kg, about 0.2×10⁶ to 0.6×10⁶ viable cells/kg, or about 0.2×10⁶ to 0.4×10⁶ viable cells/kg.

In some embodiments, the genetically engineered innate lymphoid cells are administered in a single dose or can be administered in a plurality of doses, such as given daily, every other day or every three days. For example, in some embodiments, the genetically engineered innate lymphoid cells are administered between about 1 and 5 times, such as between about 3 and 5 times. In some embodiments, plurality of doses are administered on the same day, such as 1 to 5 times or 3 to 5 times daily.

Without further description, it is believed that one of ordinary skill in the art can, using the preceding description and the following illustrative examples, make and utilize the innate lymphoid cells of the present disclosure and practice the claimed methods. The following working examples therefore, specifically point out the preferred embodiments of the present disclosure, and are not to be construed as limiting in any way the remainder of the disclosure.

### EXAMPLE

### Materials and Methods

### Isolation of NK Cells

The transgenic mouse carrying EKLF K74R mutant allele was generated in according to procedures described in WO 0367272016. WT and Eklf (K74R) mice were sacrificed by cervical dislocation and the spleens were isolated. Spleens were dissociated into single cells by using the gentle MACS C tubes and the dissociator following the manufacturer's protocol (MACS Miltenyi Biotec.). Cell pellets were resuspended by RBC lysis buffer and centrifuged at 2,000 rpm for 4 minutes immediately. Anti-mouse antibodies CD3ε (PE-610), CD45R (APC), NK1.1 (V450) and Nkp46 (PE-Cy7) (eBiosciences, 61-0031-82, 17-0452-83, 48-5941-82, 25-3351-82) were used for sorting the murine splenic NK cells by flow cytometry (BD FACS Aria II SORP).

### Expansion of Mouse NK Cell Culture

Sorted NK cells were cultured at the concentration of 1×10⁶ cells/mL in RPMI medium containing 10% FBS, 2% P/S, 2-ME (50 μM). During expansion, the cells were provided with IL-15 (50 ng/ml) every day. Half of the medium was replaced by fresh medium containing the corresponding cytokines. IL-15 (50 ng/ml) was added for 6 days and 7 days for in vitro cytotoxicity assay and in vivo tumor inhibition assay, respectively. IL-2 (200 U/ml) was added to boost the function of NK cells on day 5 before in vitro cytotoxicity assay.

### In Vitro Cytotoxicity Assay of NK Cells

Melanoma B16F10 cells were washed two times by PBS, and then the cells (1×10⁷ cells/ml) were stained with CFSE (1 μM) for 20 minutes. After 20 minutes, the cells were washed two times by RPMI medium and seeded into 96-well v-bottom plate (0.3×10⁵ cells/well). After 6 days of expansion and prime by IL-2, the NK cells (effector cells) were co-culture with CFSE-labeled B16F10 (target cells) at different ratios of effector cells (E) and target cells (T) for 4 hours. The cells were then stained with PI and analyzed for the extents of cancer cell lysis by flow cytometry.

### In Vivo Assay of Inhibition of Tumor Growth

B16F10-Luc cells (4×10⁴ cells/mice) were injected intravenously (i.v.) in C57BL/6 mice. When the bioluminescence signals were detected on the lung or liver in the cancer-bearing mice on day 4, the mice were injected with the expanded WT NK and NK (K74R) cells (2×10⁶ cells/mouse), respectively. The bioluminescence signals were evaluated by in vivo image system (IVIS) on day 4, 7, 11, and 14 post-tumor cell injection.

### Example 1: In Vitro Cancer Cell Killing Capability of Mouse NK (K74R) Cells

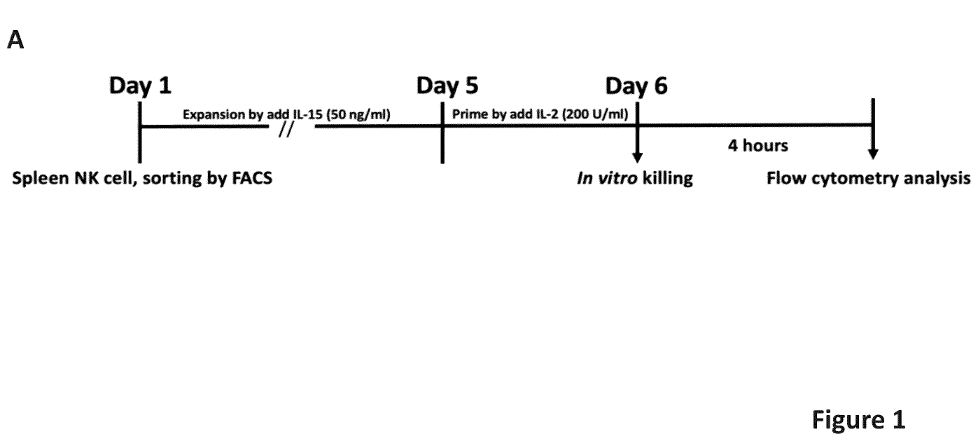

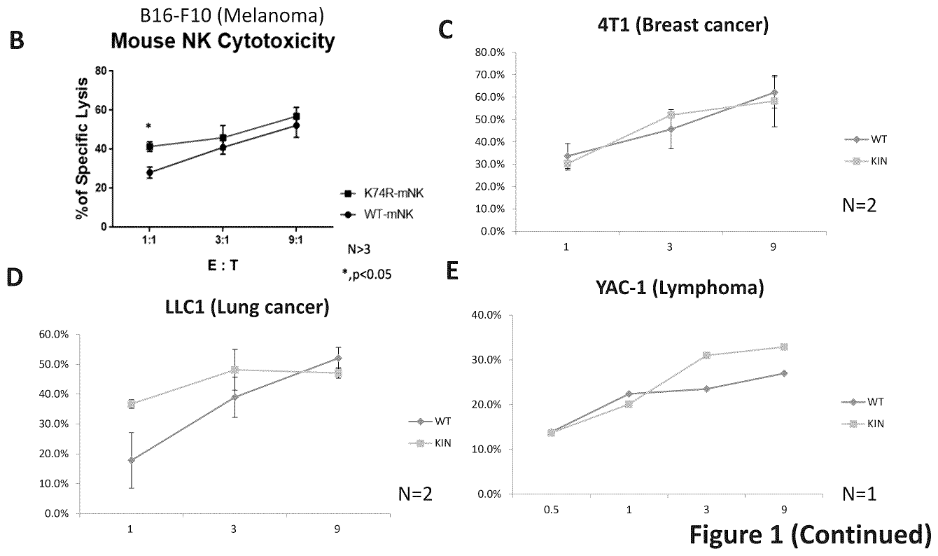

In order to investigate the detail mechanisms of the anti-cancer capability of the Eklf (K74R) mice, the NK cells were isolated from WT and Eklf (K74R) splenocytes, respectively (FIG. 1A), and were performed the in vitro cytotoxicity assay to compare their abilities to kill B16F10 melanoma cells in vitro. FIGS. 1B to 1D showed the lysis in four different cancer cell lines. NK (K74R) cells showed higher cancer cell cytotoxicity in B16-F10 (FIG. 1B) and LLC1 (FIG. 1D) than WT NK cells, especially at the ET ratio of 1:1, and showed higher cancer cell cytotoxicity in YAC-1 (FIG. 1E) at the E:T ratio of 9:1. The data suggests that NK cells contribute to better broad anti-cancer character of the Eklf (K74R) mice.

### Example 2: Capability to Inhibit Tumor Growth In Vivo by Mouse Eklf (K74R) NK Cells

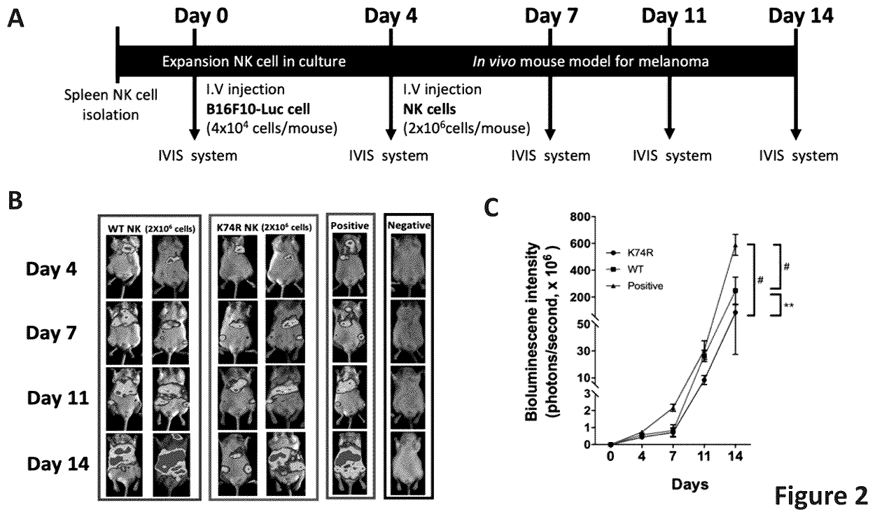

To compare the capabilities of the NK (K74R) and WT NK cells to inhibit tumor growth, mouse NK cells (NK1.1⁺, Nkp46⁺) were sorted from splenocytes, and were expanded by using IL-15 (50 ng/ml) for 7 days. As shown in FIG. 2A, cultured B16-F10 cancer cells were injected into the NSG mice on day 0. Expanded mouse NK cells (2×10⁶/mouse) were injected into B16-F10 cancer-bearing mice on day 4. After tail vein injection with NK cells, the mice were subjected to in vivo imaging system by IVIS on day 4, 7, 11, and 14. As shown in FIGS. 2B and 2C, the melanoma B16-F10-Luc tumor foci formed on the lung and liver on day 4. The bioluminescent intensities were similar in WT NK cell-treated and NK (K74R) cell-treated mice on day 7; however, the tumor growth was significantly slower in the NK (K74R) cell-treated mice than WT NK cell-treated ones on day 11 and beyond, as shown in FIGS. 2B and 2C.

### Example 3: Administration of Bone Marrow Mononuclear Cells (BMMNCs) (K74R) Prolongs the Lifespan of Recipient Wild-Type Mice

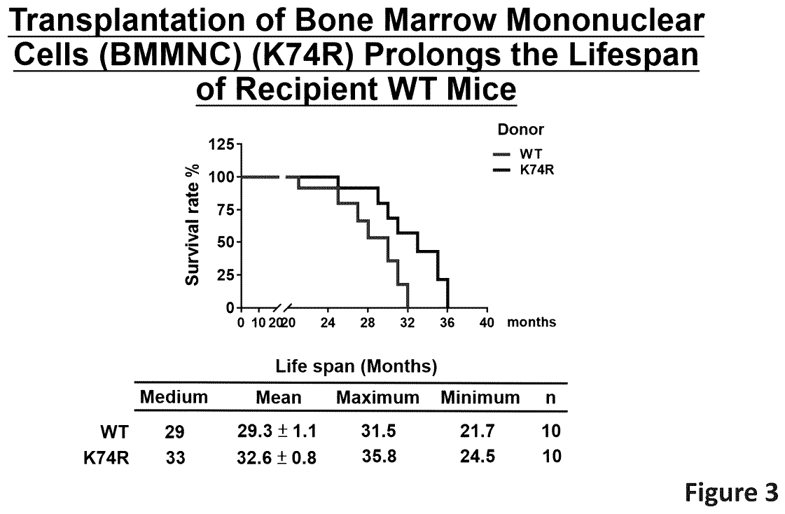

The bone marrow mononuclear cells of 2 month-old WT or Eklf (K74R) mice were injected into the vein of 2 month-old WT recipient mice. The medium survival curve of mice receiving BMMNC from WT mice is 29 months, while that of mice receiving BMMNC transplantation from the Eklf (K74R) mice is 33 months, as shown in FIG. 3. The data suggests that BMMNCs including NK cells (K74R) prolongs the lifespan of recipient wild-type mice.

What is claimed is:

1. A method of increasing longevity and/or treating cancer in a subject, comprising administering an effective amount of genetically engineered innate lymphoid cells to the subject, wherein the genetically engineered innate lymphoid cells express a modified EKLF polypeptide comprising an amino acid substitution at a sumoylation site of a wild-type EKLF polypeptide having the amino acid sequence of SEQ ID NO: 1 or SEQ ID NO: 2.

2. The method of claim 1, wherein the genetically engineered innate lymphoid cells are natural killer (NK) cells.

3. The method of claim 1, wherein the genetically engineered innate lymphoid cells are prepared by the steps of:
(a) providing a transgenic animal expressing the modified EKLF polypeptide;
(b) isolating innate lymphoid cells from the transgenic animal; and
(c) expanding the innate lymphoid cells with a cytokine selected from the group consisting of IL-2, IL-12, IL-15, IL-18 and IL-21; and optionally
(d) contacting the innate lymphoid cells with an irradiated feeder cells.

4. The method of claim 1, wherein the sumoylation site corresponds to lysine at position 54 of a wild type human EKLF, or corresponds to lysine at position 74 of a wild type mouse EKLF.

5. The method of claim 1, further comprising administering long-term hematopoietic stem cells (LT-HSC) expressing a modified EKLF polypeptide to the subject, wherein the modified EKLF polypeptide comprises a substitution of K54R in the wild type human EKLF, or a substitution of K74R in the wild type mouse EKLF.

6. The method of claim 2, wherein
(i) the NK cells are CD56^{bright} NK cells expressing CD62L, CCR7 and CXCR4;
(ii) the NK cells are CD56^{dim} NK cells expressing CXCR1, CX3CR1 and ChemR23;
(iii) the NK cells express NK1.1 (CD161) and NKp44 or Nkp46;
(iv) the NK cells express CD49b; or
(v) the NK cells express CD45 and CD56.

7. The method of claim 1, wherein
(i) the innate lymphoid cells do not express CD117, Sca-1, and CD90.1 (Thy1.1);
(ii) the innate lymphoid cells express CRTH2, KLRG1, SST2, CD25, CD44 and CD161;
(iii) the innate lymphoid cells express RORγt, NKp30 and CD56; or
(iv) the innate lymphoid cells express c-Kit, CCR6, CD25, CD90, CD127, and OX40L.

8. The method of claim 1, wherein the modified human EKLF polypeptide comprises a substitution of the lysine (K) residue corresponding to position 54 of the wild type human EKLF with an arginine (R) or with another amino acid that confers tumor resistance and healthy longevity.

9. The method of claim 1, wherein the modified EKLF polypeptide is a modified mouse EKLF polypeptide comprises an amino acid substitution at position 68 of the full length wild-type mouse EKLF polypeptide.

10. The method of claim 1, wherein genetically engineering innate lymphoid cells comprise transducing the innate lymphoid cells with a viral vector encoding the modified EKLF polypeptide.

11. The method of claim 1, wherein genetically engineering innate lymphoid cells comprise using clustered regularly interspaced short palindromic repeats (CR1SPR) or CR1SPR associated proteins (Cas) technique.

12. The method of claim 1, comprising administering 5×10⁶ to 5×10⁸ genetically engineered innate lymphoid cells per kilogram to the subject.

13. The method of claim 1, wherein the cancer is liver cancer, colon cancer, breast cancer, prostate cancer, hepatocellular carcinoma, skin cancer, lung cancer, glioblastoma, brain cancer, hematopoietic malignancy, retinoblastoma, renal cell carcinoma, head and neck cancer, cervical cancer, pancreatic cancer, esophageal cancer, or squama cell carcinoma.

14. The method of claim 1, wherein the cancer is melanoma, breast cancer, lung cancer or lymphoma.

15. An in vitro method of producing genetically engineered innate lymphoid cells, comprising:
(a) providing a transgenic animal expressing a modified human EKLF polypeptide comprising an amino acid substitution at the sumoylation site that corresponds to lysine at position 54 of the wild type human EKLF having the amino acid sequence of SEQ ID NO: 1, or a modified mouse EKLF polypeptide comprising an amino acid substitution at the sumoylation site that corresponds to lysine at position 74 of the wild type mouse EKLF having the amino acid sequence of SEQ ID NO:2; and
(b) isolating innate lymphoid cells from the genetically engineered animal.

16. The method of claim 15, further comprising expanding the innate lymphoid cells with a cytokine selected from the group consisting of IL-2, IL-12, IL-15, IL-18 and IL-21.

17. The method of claim 15, further comprising depleting CD3⁺ cells using magnetic beads coated with an anti-CD3 antibody.

18. The method of claim 15, further comprising contacting the innate lymphoid cells with an irradiated feeder cells.

19. The method of claim 18, wherein the irradiated feeder cells are EBV-transformed lymphoblastoid cell lines, or leukemia cell lines modified to express a membrane-bound form of IL-15 and 41BB ligand.

20. An engineered innate lymphoid cell comprises a gene encoding a modified EKLF polypeptide, wherein the modified EKLF polypeptide comprises an amino acid substitution at a sumoylation site of a wild-type EKLF polypeptide, wherein the sumoylation site corresponds to lysine at position 54 of a wild type human EKLF having the amino acid sequence of SEQ ID NO: 1, or corresponds to lysine at position 74 of a wild type mouse EKLF having the amino acid sequence of SEQ ID NO: 2.


SEQUENCE LISTING (2 sequences)

SEQ ID NO: 1 - protein, 362 aa, Homo sapiens
MATAETALPSISTLTALGPFPDTQDDFLKWWRSEEAQDMGPGPPDPTEPPLHVKSEDQPGEEEDDERGADATWDLDLLLTNFSGPEPGGAPQTCALAPSEASGAQYPPPPETLGAYAGGPGLVAGLLGSEDHSGWVRPALRARAPDAFVGPALAPAPAPEPKALALQPVYPGPGAGSSGGYFPRTGLSVPAASGAPYGLLSGYPAMYPAPQYQGHFQLFRGLQGPAPGPATSPSFLSCLGPGTVGTGLGGTAEDPGVIAETAPSKRGRRSWARKRQAAHTCAHPGCGKSYTKSSHLKAHLRTHTGEKPYACTWEGCGWRFARSDELTRHYRKHTGQRPFRCQLCPRAFSRSDHLALHMKRHL

SEQ ID NO: 2 - protein, 376 aa, Mus musculus
MRQKRERRPEVQGGHQPAMASAETVLPSISTLTTLGQFLDTQEDFLKWWRSEETQDLGPGPPNPTGPSHHVSLKSEDPSGEDDERDVTCAWDPDLFLTNFPGSESPGTSRTCALAPSVGPVAQFEPPESLGAYAGGPGLVTGPLGSEEHTSWAHPTPRPPAPEPFVAPALAPGLAPKAQPSYSDSRAGSVGGFFPRAGLAVPAAPGAPYGLLSGYPALYPAPQYQGHFQLFRGLAAPSAGPTAPPSFLNCLGPGTVATELGATAIAGDAGLSPGTAPPKRSRRTLAPKRQAAHTCGHEGCGKSYTKSSHLKAHLRTHTGEKPYACSWDGCDWRFARSDELTRHYRKHTGHRPFCCGLCPRAFSRSDHLALHMKRHL